(19) United States
(12) Patent Application Publication
(10) Pub. No.: US 2019/0183607 A1
(43) Pub. Date: Jun. 20, 2019

(54) A-Flex Assit Arm

(71) Applicants: Aurelia Byrne, Plantation, FL (US); Gary Slowinski, Plantation, FL (US)

(72) Inventors: Aurelia Byrne, Plantation, FL (US); Gary Slowinski, Plantation, FL (US)

(21) Appl. No.: 15/844,508

(22) Filed: Dec. 15, 2017

(51) Int. Cl. A61C 1/14; A61B 50/20

(52) U.S. Cl. CPC A61C 1/145; A61C 17/04; A61B 50/20

(57) ABSTRACT

The a-flex assist arm is a device that attaches to any surface on a dental chair or hospital bed or in either case attachment to any surrounding surfaces in the dental office or any medical setting. It is designed to hold any suction device but not limited to just holding a medical or dental suction. The A-flex is essentially a third arm that can be used in any field holding items such as tools, instruments or devices.

### BACKGROUND

The A-flex is designed as a flexible assistant arm to hold a suction device such as saliva ejector or any evacuating extraction device in the medical or dental field. The A-Flex has been designed to help the dental hygienist by holding the saliva ejector in place while working. A hands free assisting mechanism allowing the operator to utilize proper ergonomics. No longer does the patient have to hold the suction or have the suction draping off the side of his or her cheek. This also allows the dental hygienist to maintain a proper ergonomic position preventing muscular skeletal issues. In the medical field such as a hospital setting the A-Flex can be used to hold a evacuating instrument to aspirate saliva or sputum from a patient that is quadriplegic. The A-flex is a third arm that can attach anywhere or be connected or built in to any medical or dental furniture or clinical chairs or auxiliary equipment. The suction cup device can attach to any surface by means of direct contact or a removable docking station or it can be built in to any dental chair dental unit or any medical equipment. In either or field dental or medical it is designed to be used as a third arm to assist both patient and practitioner.

### DETAILED DESCRIPTION OF THE INVENTION

The A-Flex is a flexible arm that can be attached to any dental chair or medical equipment that is plastic metal or any substance that is solid. The flexible arm is made from any or all-flexible high grade medical or industrial materials including any metal, plastic, composite, composite resin, wires, stainless steel, aluminum, titanium, rubber, wood, coils, PVC pipe solid or flexible. The arm of the A-Flex has an attached suction device or detachable docking station device or any sort of clamp, composite, adhesive materials and or any welded aluminum, tin, stainless steel, soldering metals, rubber, plastic, or suction cups. Both the Removable docking station or non-removable docking station is attached to any where on the dental chair the back, front, sides, chair arms, between cushions, over-head light or foot of the chair, base of the chair or any auxiliary dental chairs or removable equipment, and counter tops. Also the A-Flex assist arm attaches via removable means with materials such as clamps, magnets, adhesives, composites, suctions, any applied pressure force or pressurized air source. The coating of the A-Flex arm is surrounded or incased with a medical grade and or industrial grade tape, adhesive, rubber material or acrylic, oil or water-based paint.

The A-flex assist arm holds the dental saliva ejector or any suction with a metal clamp, plastic clamp, or any high-grade stainless steel or industrial high-grade medical clamps. The A-Flex arm has an interchangeable joint or ball hinge joint that swivels, rotates for better mobility of the saliva ejector or any other suction device. The ball hinge joint is either on the arm itself or on the removable docking station or built in docking station. Finally the A-Flex is designed to have detachable interchangeable parts to allow ease for multiple users and efficiency.

### DESCRIPTION OF DRAWINGS

FIG. 1 Is the A-flex assist arm in entirety.

FIG. 2 Any suction, vacuum, clip, clamp, magnet or strap on to any chair or equipment.

FIG. 3 90-degree elbow that connects flexible gooseneck, (FIG. 4) to mounting device (FIG. 2), Made with any durable material.

FIG. 4 Flexible metal, steel, carbon fiber or plastic, with both ends interchangeable for length or flexible strength. This could eventually attach to any chair or equipment.

FIG. 5 90-degree elbow with any clip or clamp for holding a saliva ejector any other device or tissue. May be flexible or solid material. This attaches to the flexible gooseneck (FIG. 4)

FIG. 6 Any device that may be attached and serve as a better mounting surface. This will have an adhesive reusable back and will have a different diameter than (FIG. 2)

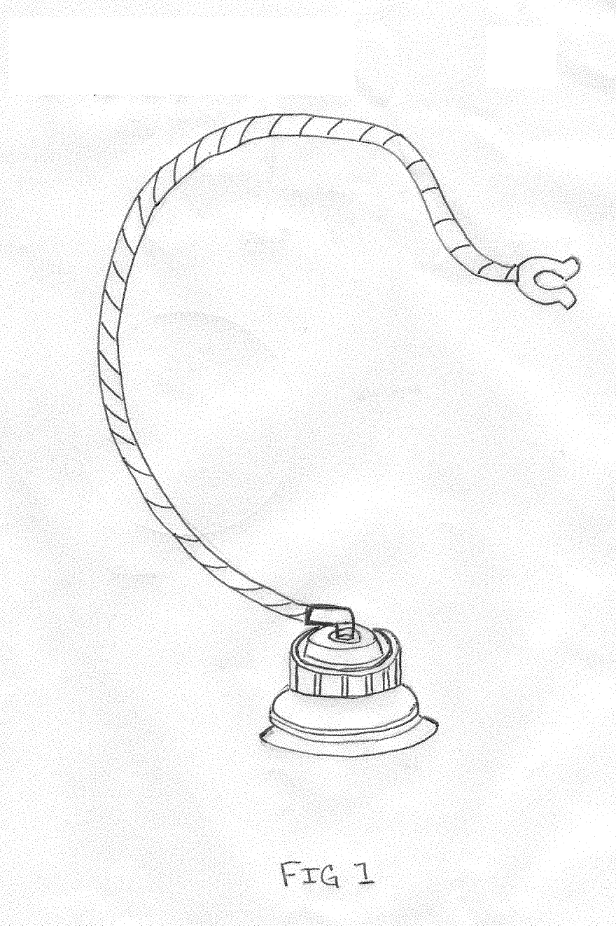

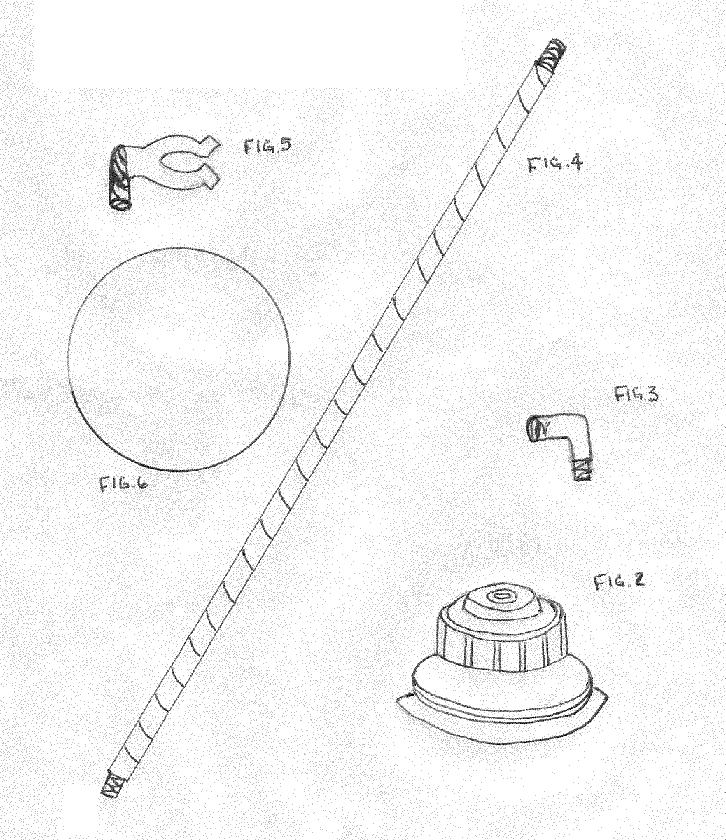

The invention claim is:

1. The A-Flex assist arm can be attached to: a. a dental chair or any medical equipment via a suction device or, b. a circular removable docking station with a smooth surface on one side and adhesive on the opposite side, c. a removable docking station is to adhere to any surface that is not attachable via the suction device on the base of the A-Flex, d. at the base is an attachment that is a flexible material with attached clip, e. a dental or medical suction is held with attached clip, f. the A-Flex assist arm is designed to have detachable interchangeable parts to allow ease for multiple users and efficiency.

2. The A-Flex assist arm of claim 1 houses a flexible arm that a clip is attached to.

3. The A-Flex assist arm of claim 1 houses a clip that is used to hold a dental or medical suction, instruments or devices.

4. The A-Flex assist arm of claim 1 flexible arm and is attached to a suction cup that allows for attachment to a dental chair or medical equipment.

5. The A-Flex assist arm of claim 1 has a docking station that is circular that has adhesion on one side and a flat smooth surface, to house the suction cup.

6. The A-Flex assist arm of claim 2 houses the flexible arm made of any material that is flexible.

7. The A-flex assist of claim 6 the flexible arm can be made by any means such as mechanical or robotic.

8. The A-Flex assist arm of claim 3 houses a clip that holds any dental or medical suction device this clip is made out of different materials and shapes.

9. The A-Flex assist arm of claim 3 attaches via suction to a dental chair but can also be built into a dental chair or any medical or dental equipment or wheelchair.

10. The A-Flex assist arm of claim 1 flexible arm, clip can withstand sterilization by heat and pressure as well as any disinfectant products.

11. The A-flex assist arm of claim 5 has an adhesive circular docking station that can be reused and moved from multiple dental chairs or medical equipment.

12. The A-Flex assist arm of claim 2 Flexible arm can be detached from clip allowing for accessory clip and arm attachments.

13. The A-Flex assist arm of claim 1 Can be built in to any dental chair or medical equipment or wheel chair.

14. The A-Flex assist arm of claim 7 The arm of the A-Flex can be a robotic or self moving.

15. The A-Flex assist arm of claim 12 The flexible arm can be adjusted at the base for all directional positioning.

16. The A-Flex assist arm of claim 3 The clip attached to the flexible arm can be adjusted for all directional positioning.

17. The A-flex assist arm of claim 1 will have an adjustable light source either attached as an extension or built into flexible arm.
